(19) United States
(12) Patent Application Publication
(10) Pub. No.: US 2020/0190556 A1
(43) Pub. Date: Jun. 18, 2020

(54) METHOD FOR DETECTING ACTIVITY OF THIOREDOXIN REDUCTASE, DETECTION DEVICE AND OPERATION METHOD THEREFOR

(71) Applicants: NANJING KEAISE MEDICINE & TECHNOLOGY CO. LTD., Nanjing, Jiangsu (CN); Wuhan Shangyi Health Science & Technology Corporation, Wuhan, Hubei (CN); Keaise Medicine Wuhan Corp., Wuhan, Hubei (CN)

(72) Inventor: Hanwei YIN, Nanjing, Jiangsu (CN)

(21) Appl. No.: 16/496,207

(22) PCT Filed: Mar. 21, 2018
(86) PCT No.: PCT/CN2018/079787

§ 371 Date: Jan. 2, 2020

(30) Foreign Application Priority Data
Mar. 21, 2017 (CN) 201710170517.9
Mar. 21, 2017 (CN) 201710172659.9

(51) Int. Cl. C12Q 1/26; G01N 1/28; G01N 35/04; G01N 35/00; G01N 35/10

(52) U.S. Cl. CPC C12Q 1/26; G01N 1/28; G01N 35/04; G01N 2035/0441; G01N 35/1004; G01N 35/1009; G01N 2035/0401; G01N 35/00722

(57) ABSTRACT

The present invention discloses a method for detecting activities of a thioredoxin reductase, a detection device and an operation method therefor. The detection method comprises: solution preparation: preparing a working solution, an inhibitor solution, and a mixed agent; sample addition: adding the working solution into a control reaction cup, adding the inhibitor solution into an experimental reaction cup, and respectively adding a sample into the control reaction cup and the experimental reaction cup; incubation: putting the control reaction cup and the experimental reaction cup in a dark environment to perform incubation at a predetermined temperature for a first predetermined time; and measurement: adding the mixed agent into the control reaction cup and the experimental reaction cup and measuring the absorbance value at a predetermined wavelength for a second predetermined time. Said detection method and the detection device can achieve fully automated detection of activities of the thioredoxin reductase, being fast and efficient, and saving time and effort.

### TECHNICAL FIELD

The present invention belongs to the field of enzyme activity detection, specifically relates to a biochemical detection method for thioredoxin reductase (TR) activity in human blood, a detection apparatus and an operation method thereof.

### BACKGROUND ART

Thioredoxin reductase (TR) detection project is the first clinical tumor detection project at home and abroad. This project can fill in the lack of clinical detection methods in the diagnosis of dysplastic diseases.

TR detection apparatus can be applied in early screening of cancers in people of medical examination, monitoring tumor efficacy in inpatients, early warning of recurrence and health management, etc., which has great market demand and development potential.

The prior art disclosed by “Patent ZL201080049877. X: Methods and reagent kits for determining the activity of thioredoxin reductase and the uses thereof” is realized by being based on manual step-by-step operation, such as: manual operation of enzyme marker, manual sample adding, manual operation of avoiding light, manually placing shaking table to achieve shaking homogenously, etc.; various conditions on samples are also for manual operation process and usage design. Many problems need to be improved, such as large errors due to the manual operation including manual sample adding; intermediate errors easily produced by incoherence of various operations; and long operation time which is not good for large-scale clinical detection application, etc.

The TR assay kits in the prior art are preliminary R & D products, which have certain limitations in detection steps, movements, throughputs, etc., so there is still room for improvement and correction in detection speed and accuracy. For example, the detection efficiency is low only with manual detection of thioredoxin reductase (TR) in human blood; the data detected with a biochemical detection apparatus in the existing technology do not meet the distribution of national data indicators, and so on.

Therefore, it is urgent to propose a fully automated biochemical detection method and a detection apparatus for thioredoxin reductase (TR) to improve detection efficiency and to make the detected data to meet the distribution of national data indicators.

### SUMMARY OF THE PRESENT INVENTION

The object of the present invention is to provide a new detection method for fully automatically detecting thioredoxin reductase activity, a detection apparatus and its operation method, to save detection time and reduce detection steps. The present invention solves the problem of manual detection of thioredoxin reductase in human blood in the prior art, and can improve the detection efficiency of thioredoxin reductase in clinic and save cost. The present invention achieves for the first time the automated operation of “TR activity assay” on biochemical detection apparatus; the data directly obtained from the present invention meets the requirements of national testing standards, and ensures that the results of the detecting data reflect the early warning function of the detection technology (meeting the distribution of national data indicators).

According to one aspect of the present invention, a detection method for thioredoxin reductase activity is provided, including: preparing liquid, preparing a working solution, inhibitor solution and mixed reagent; adding samples, adding 50 μL-70 μL of said working solution to a cuvette of the control group; adding 50 μL-70 μL of said inhibitor solution to a cuvette of the test group; adding 10 μL-30 μL of a sample to the cuvette of the control group and the cuvette of the test group, respectively; wherein the amount of the samples added is designed according to the volume required for the optimal operation of the automated cooperative detection apparatus; Sample incubation, in the dark, incubating the cuvettes of the control group and the test group at 30° C.-40° C. for the first predetermined time; testing, adding 100 μL-150 μL of the mixed reagent to the cuvettes of the control group and the test group, respectively; at the predetermined wavelength, determining the absorbance values in the second predetermined time period.

Furthermore, the steps for preparing the working solution include: taking tri(hydroxymethyl)aminomethane hydrochloride, morpholinopropanesulfonic acid, disodium hydrogen phosphate/citric acid buffer and potassium dihydrogen phosphate/disodium hydrogen phosphate buffer according to the ratio of 1:1:2:4; mixing tri(hydroxymethyl)aminomethane hydrochloride, morpholinopropanesulfonic acid, disodium hydrogen phosphate/citric acid buffer and potassium dihydrogen phosphate/disodium hydrogen phosphate buffer homogeneously for the automated cooperative detection apparatus.

Furthermore, said tri(hydroxymethyl)aminomethane hydrochloride for the automated cooperative detection apparatus has a pH of 5.5-7.2, and a concentration of 0.025-0.125 mol/L; said morpholinopropanesulfonic acid for the automated cooperative detection apparatus has a concentration of 0.25 mol/L; said disodium hydrogen phosphate/citric acid buffer for the automated cooperative detection apparatus has a pH of 2.2-8.0 and a concentration of 0.2 mol/L; said potassium dihydrogen phosphate/disodium hydrogen phosphate buffer for the automated cooperative detection apparatus has a pH of 4.9-8.2 and a concentration of 1-15 mol/L.

Furthermore, the steps for preparing the inhibitor solution for the automated cooperative detection apparatus include: mixing the working solution and the inhibitor for the automated cooperative detection apparatus in a ratio of 1:1-1:5 to form the inhibitor solution for the automated cooperative detection apparatus; mixing the inhibitor solution for the automated cooperative detection apparatus homogenously, wherein, the inhibitor for the automated cooperative detection apparatus is a thioredoxin reductase inhibitor compound.

Furthermore, the steps for preparing the mixed reagent for the automated cooperative detection apparatus include: mixing reagent A for the automated cooperative detection apparatus and reagent B for the automated cooperative detection apparatus to form the mixed reagent for the automated cooperative detection apparatus in a ratio of 1:4-1:8; mixing the reagent for the automated cooperative detection apparatus homogenously; reagent A for the automated cooperative detection apparatus is 5,5′-dithiobis(2-nitrobenzoic acid) or substituted 6,6′-dinitro-3,3′-dithiobenzoic acid; and reagent B for the automated cooperative detection apparatus is nicotinamide adenine dinucleotide phosphoric acid.

Furthermore, the predetermined temperature for the automated cooperative detection apparatus is 30° C.-40° C.

Furthermore, the first predetermined time for the automated cooperative detection apparatus is 8-20 minutes.

Furthermore, the first predetermined time for the automated cooperative detection apparatus is 10 minutes.

Furthermore, the first predetermined wavelength for the automated cooperative detection apparatus is 405 nm-450 nm.

Furthermore, the second predetermined time for the automated cooperative detection apparatus is 20-30 cycles.

The present invention provides a detection method for thioredoxin reductase activity in human peripheral blood, by automatically sampling and mixing reagent A and reagent B in the automated cooperative detection apparatus to be a mixed reagent, and then automatically carrying out operations of adding it into the samples, mixing, and stirring in the automated cooperative detection apparatus, replacing the work of manually and repeatedly mixing and stirring the reagent A and reagent B which are separately added, thus improving work efficiency. By setting the sample/reagent volumes in the above automated cooperative detection apparatus which is applicable for thioredoxin reductase activity, the detection method can meet the requirements of the method for selecting the working liquid.

When the method of the present invention is applied in the above automated cooperative detection apparatus for thioredoxin reductase activity, it includes specified intelligent introduction methods for the driving system such as a method for operating sampling, and a method for the operational requirement of light shielding, a method for the operational requirement of mixing reagents, etc.

Wherein the intelligent instructions such as the number of cycles in each group of cycles and the time of each cycle specified in operation of the cooperative detection apparatus, and the requirements of the operation process are all related to the detection of TR function in human peripheral blood. The method of the present invention is a method applicable to the above cooperative detection apparatus for thioredoxin reductase activity for realizing the functional requirements of TR enzyme detection, and a detection method for thioredoxin reductase activity used in the above cooperative detection apparatus for thioredoxin reductase activity.

According to another aspect of the present invention, a type of biochemical detection apparatus for thioredoxin reductase activity is provided, including:

a holding device, which is used to hold multiple reagents and/or samples, driven by the driving device, and periodically rotated around an axis, so that the target reagents and/or samples are rotated to the target liquid-filling holes;

a reaction device, which is used to hold multiple cuvettes, driven by the driving device, and periodically rotated around an axis, so that the target cuvettes are rotated to the target sampling holes; a sampling device, which is driven by the driving device, periodically rotated around an axis and used for adding the target reagents and/or samples collected from the target sampling holes to the target cuvettes corresponding to the target liquid-filling holes;

a status sensing device, which is used for detecting the information of the reaction rotation status in the reaction device, the rotation status information of the holding in holding device, and the reagent status information in the sampling device;

a main control system, which is connected with the status sensing device and the driving device, respectively, and used for generating and sending corresponding control instructions to the driving device based on the information of the reaction rotation status, the rotation status of the holding, and the reagent status;

a driving device, which is connected with the sampling device, the holding device and the reaction device, respectively, and used for controlling the sampling device, the reaction device and the holding device to perform corresponding operations based on the received control instructions.

According to another aspect of the present invention, an operation method of the biochemical detection apparatus is provided, including:

the status sensing device, when detecting that the target cuvettes are rotated to the target liquid-filling holes and the reagent status information that the collection of reagents is completed in the reaction device, generates an instruction of the addition of reagents;

the driving device, after receiving the instruction to add reagents, controls the sampling device to rotate to the target liquid-filling holes in the reaction device and to add the target reagents and/or the target samples to the target cuvettes corresponding to the target liquid-filling holes;

the status sensing device, when detecting that the target cuvettes are rotated to the target liquid-filling holes and the reagent status information that the addition of reagents is completed in the holding device, generates the instruction for the collection of reagents;

the driving device, after receiving the instruction for the collection of reagents, controls the sampling device to rotate to the target sampling holes in the holding device and to collect the target reagents and/or the target samples.

As mentioned above, the present invention provides a fully automated biochemical detection apparatus for thioredoxin reductase (TR) in human blood, achieving full automation of the biochemical detection apparatus as well as improving detection efficiency and saving cost.

### BRIEF DESCRIPTION OF THE FIGURES

FIG. 1 is a flow chart of the detection method of the present invention for thioredoxin reductase activity;

FIG. 2 is a schematic diagram of the corresponding detection apparatus for the detection method for thioredoxin reductase activity of the present invention.

FIG. 3 is a flow chart of the detection method for thioredoxin reductase activity in a specific embodiment of the present invention.

FIG. 4 is another flow chart of the detection method for thioredoxin reductase activity in a specific embodiment of the present invention.

FIG. 5 is a flow chart of step S1 shown in the flow chart of FIG. 4.

FIG. 6 is a flow chart of step S2 shown in the flow chart of FIG. 4.

FIG. 7 is a flow chart of step S3 shown in the flow chart of FIG. 4.

FIG. 8 is a schematic diagram of the system architecture of the biochemical detection apparatus for thioredoxin reductase (TR) of the present invention.

FIG. 9 is a schematic diagram of the mechanical structure of the biochemical detection apparatus for thioredoxin reductase (TR) of the present invention.

FIG. 10 is a flow chart of the operation method of the biochemical detection apparatus for thioredoxin reductase (TR) in the tenth embodiment of the present invention.

FIG. 11 is a flow chart of the operation method of the biochemical detection apparatus for thioredoxin reductase (TR) in the eleventh embodiment of the present invention.

FIG. 12 is a flow chart of the operation method of the biochemical detection apparatus for thioredoxin reductase (TR) in the twelfth embodiment of the present invention.

FIG. 13 is a flow chart of the operation method of the biochemical detection apparatus for thioredoxin reductase (TR) in the thirteenth embodiment of the present invention.

FIG. 14 is a schematic diagram of the detection principle of the biochemical detection apparatus in a specific example in the present invention.

10. Holding device, 11. Holding disk, 12. Holding fixture, 20. Reaction device, 21. Reaction disk, 22. Fixtures for cuvettes, 30. Sampling device, 31. Sampling rotating unit, 32. Sampling fixtures, 33. Sampling needle, 40. Stirring device, 41. Stirring rotating unit, 42. Stirring fixtures, 43. Stirring needle, 50. Status sensing device, 60. Main control system, 70. Driving device, 80. Temperature control device, 90. Cleaning device, 91. Hydraulic device, 92. Cleaning component, 100. Power system, 101. First power subsystem, 102. Second power subsystem, 103. Third power subsystem, 104. Fourth power subsystem, 110. Client.

### EXAMPLES

Hereinafter, the present invention will be further illustrated in more detail below with reference to embodiments to make the objects, technical solutions and technical effects clearer. It is to be understood that the examples described in the description are only illustrative of the present invention and are not intended to limit the present invention. Furthermore, in the following description, the description of known structures and techniques is omitted to avoid unnecessarily obscuring the concepts of the present invention.

There are three technical limitations in the detection method for “TR Activity Assay Kit” (hereinafter referred to as the preliminary detection method) of the prior art, so it cannot be applicable to automated cooperative detection apparatus:

(1) long time consuming and low detection efficiency. The preliminary detection method allows 8-12 samples to be detected at the same time, and the time for completion is about 1.5-2 hours. The automated cooperative detection apparatus for thioredoxin activity requires that every 40-50 samples are detected within 1.5 hours, otherwise the clinical samples will invalid due to the long time in the apparatus; therefore, the preliminary detection method cannot be applied for the large-scale, high-throughput TR clinical automated detection;

(2) numerous detection steps. The preliminary detection method includes seven steps and movements, which is not difficult for testers to detect manually, but too complicated for the automated cooperative detection apparatus, so it significantly increases the operation time of the apparatus and reduces the detection efficiency;

(3) in the process of operation, two testers are required to cooperate with each other in the preliminary detection method, while automated cooperative detection apparatus is designed to allow a single tester to operate the apparatus completely, so the requirements of the two methods are not consistent.

Before elaborating on the embodiments of the present invention, it should be noted that reagent A in the present invention is a reagent for detecting thioredoxin reductase activity, reagent B is a reagent for detecting thioredoxin reductase activity, and the working solution is usually a buffer solution, which is mainly used for detecting thioredoxin reductase activity.

Reagent A and reagent B in the present invention, both of which have passed the expert certification examination administrated by the China Food and Drug Administration, and obtained Registration Certificate for Medical Device (certificates: Hubei Food and Drug Administration (approval) [2013] No. 2401815 and China Food and Drug Administration (approval) [2014] No. 3400264), are reagent A and reagent B in “Thioredoxin Reductase (TR) Activity Assay Kit”, the working solution is reagent D in the above “Thioredoxin Reductase (TR) Activity Assay Kit”, and the inhibitor in the present invention is reagent C in the above “Thioredoxin Reductase (TR) Activity Assay Kit”.

Thioredoxin reductase (TR), which is a reduced coenzyme II (NADPH)-dependent, and flavin adenine dinucleotide (FAD)-containing dimeric selenoenzyme, forming a thioredoxin system together with thioredoxin, and reduced coenzyme II. Thioredoxin reductase is overexpressed in cells with abnormally active proliferation, and it has physiological functions such as initiating abnormal cell proliferation and activating apoptosis inhibition system, etc., which are closely related to tumor formation. TR activity is highly correlated with the degree of abnormal proliferation of tumors. Therefore, the detection of thioredoxin reductase plays an important role in the tumor detection.

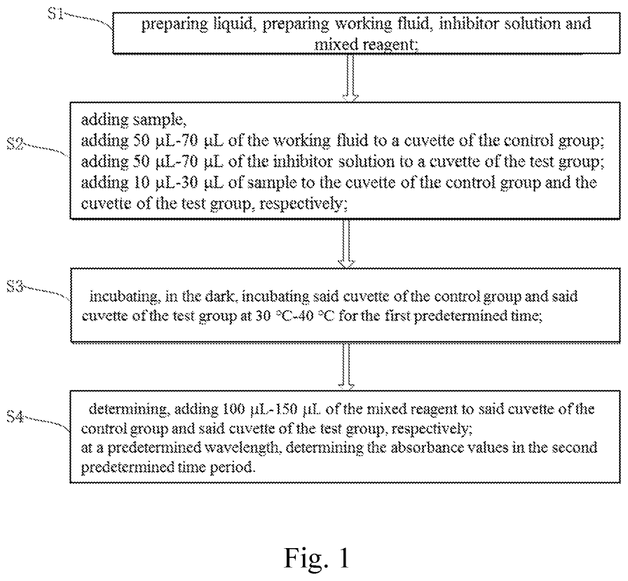

FIG. 1 is a flow chart of the detection method of the present invention for thioredoxin reductase activity.

As shown in FIG. 1, the detection method for thioredoxin reductase activity included: Step S1, preparing liquid, comprising preparing working solution, inhibitor solution and mixed reagent;
specifically, the working solution is usually a buffer, of which the concentration is not particularly limited, and which is preferably prepared in a concentration of the 1×.

The steps for preparing the inhibitor solution and mixed reagent are as follows:

preparing 1.67 mg/mL reagent A;

preparing 10.29 mg/mL reagent B;

preparing the working solution;

wherein, the preparation process of the working solution is: taking tri(hydroxymethyl)aminomethane hydrochloride (TrisHCl) (0.025-0.125 mol/L, pH 5.5-7.2), morpholinopropanesulfonic acid (0.25 mol/L), disodium hydrogen phosphate/citric acid buffer (0.2 mol/L) and potassium dihydrogen phosphate/disodium hydrogen phosphate buffer (1-15 mol/L) according to the ratio of 1:1:2:4; wherein, the pH of disodium hydrogen phosphate/citric acid buffer is 2.2-8.0; the pH of potassium dihydrogen phosphate/disodium hydrogen phosphate buffer is 4.9-8.2; then mixing tri(hydroxymethyl)aminomethane hydrochloride (TrisHCl), morpholinopropanesulfonic acid, disodium hydrogen phosphate/citric acid buffer and potassium dihydrogen phosphate/disodium hydrogen phosphate buffer homogeneously.

preparing the working solution;

specifically, mixing the working solution and an inhibitor in a ratio of 1:1-1:5 to form the inhibitor solution; mixing the inhibitor solution homogenously; wherein, the inhibitor is a thioredoxin reductase inhibitor compound, which can be a chemical monomer such as selens.

Among them, the mixing ratio of the working solution and the inhibitor is preferably 1:3, which is the most economical ratio, that is, it can be more accurate for subsequent detection of thioredoxin reductase activity in human peripheral blood, and it is also the most economical ratio for the combination of various reagents.

Mixing the reagent A and the reagent B to form the mixed reagent in a ratio of 1:2-1:8; specifically, the range of the mixing ratio of reagent A and reagent B is 1:2-1:8, preferably 1:4-5, at which the mixed reagent formed by mixing for the detection provides higher accuracy, wherein, the reagent A is 5,5′-dithiobis (2-nitrobenzoic acid) or substituted 6,6′-dinitro-3,3′-dithiobenzoic acid; the reagent B is nicotinamide adenine dinucleotide phosphoric acid.

Mixing the working solution homogeneously; mixing the inhibitor solution homogeneously; and mixing the mixed reagent homogeneously. Specifically, the homogeneously mixed working solution, inhibitor solution and mixed reagent are placed separately, usually in the reagent groove for the subsequent detection.

Step S2, adding sample, adding 50 μL-70 μL of the working solution to a cuvette of the control group; adding 50 μL-70 μL of the inhibitor solution to a cuvette of the test group; adding 10 μL-30 μL of the sample to the cuvette of the control group and the cuvette of the test group, respectively; wherein the cuvettes of the control group and the test group are set at intervals, for example, odd numbered cuvettes are the cuvettes of the control group, even numbered cuvettes are the cuvettes of the test group.

Specifically, the amount of the working solution added to the cuvettes of the control group and the test group is the same, in general, when detecting a sample, 50 μL-70 μL of the working solution added to the cuvettes of the control group and the test group, preferably 50 μL-60 μL. Preferably, the same amount of the working solution is added to the cuvettes of the control group and the test group, so that the detection data of the control group and the test group are comparable and the data after detection can be calculated.

When the thioredoxin reductase activity is detected with the present invention, generally 1-16 samples are tested in a group, the time for addition of the samples each group is about 10 minutes, i.e. 27 cycles, 22.5 seconds per cycle, after adding a group of samples, incubating the group of samples, and then adding another group of samples.

Step S3, incubating, in the dark, incubating the cuvettes of the control group and the cuvettes of the test group at 30° C.-40° C. for the first predetermined time; wherein, the predetermined temperature is 30° C.-40° C.; the first predetermined time is 8-20 minutes, preferably 10 minutes.

Specifically, after adding the samples, incubating the cuvettes of the control group and the cuvettes of the test group at 30° C.-40° C. in the dark for 8-20 minutes, preferably 10 minutes. As the detection method can be used in the detection apparatus, when the predetermined time is 10 minutes, the automated detection of thioredoxin reductase activity can be realized in the cooperative detection apparatus. After incubation time of a group of samples, the group of samples can be detected.

Step S4, testing, adding 100 μL-150 μL of the mixed reagent to the cuvettes of the control group and the test group, respectively; at the predetermined wavelength, determining the absorbance values in the second predetermined time period. Specifically, when testing the samples, firstly, adding 110 μL-130 μL of the mixed reagent to the cuvettes of the control group and the test group with sampling needles, preferably 120 μL of the mixed reagent, at the wavelength of 405 nm-450 nm, continually determining the absorbance values for 7.5-11.25 minutes, i.e., 20-30 cycles.

In the present invention, after mixing reagent A and reagent B, and stirring together to mix them homogeneously, during detecting, the mixed reagent is added directly to the reagent to be detected, which achieves the addition by mixing. Compared with the prior art, in which the reagent A and the reagent B are separately stirred, and then the sample is separately added, a part of the steps are omitted, the detection time is saved, and the automatic detection on the synergistic detection device of the thioredoxin reductase activity is realized with improvement of the detection efficiency. wherein, the sample in the present invention refers to any tissue from organism or part separated from it. The sample is preferably selected from blood, body fluid, tissue homogenate, preferably blood, wherein the blood can be composed of serum, plasma, etc.

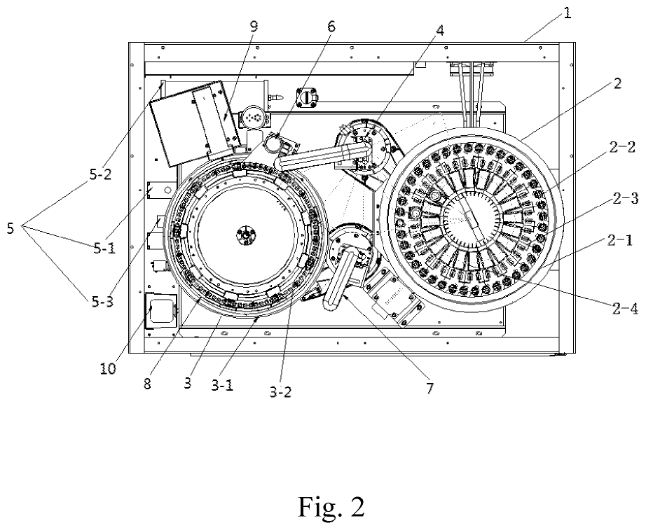

The detection method of the present invention can be applicable to “a detection apparatus for thioredoxin reductase activity”, as shown in FIG. 2, the structure of the apparatus is specifically as follows:

the detection apparatus including:

a housing 1,

a holding device 2, which is used to hold multiple reagents and samples (including working solution/inhibitor solution and mixed reagent), driven by driving device (not shown in the figure), and periodically rotated around an axis; wherein, the holding device 2 includes: a sample reagent disk 2-1, fixtures for sample tubes 2-2, fixtures for working solution/inhibitor solution 2-3, fixtures for mixed reagents 2-4, and the fixtures are uniformly distributed along the circumference direction of the sample reagent disk 2-1, respectively; the fixtures for sample tubes 2-2 can be a circle structure component with several holes arranged around the circumference direction of the sample reagent disk, and are placed in the sample reagent disk for holding sample tubes. For example, a tube holder, the number of holes in the fixtures for sample tubes is preferably 40; the structures of the fixtures for working solution/inhibitor solution 2-3 and the fixtures for mixed reagents 2-4 are similar to that of the fixture for sample tubes, the fixtures are used for holding working solution/inhibitor solution bottles or mixed reagent bottles, respectively, the number of the fixtures are preferably 30 or 40, and each fixture for working solution/inhibitor solution 2-3 and each fixture for mixed reagents 2-4 are all centered on the center of the sample reagent disk, and distributed in turn from inside to outside along the radius of the sample reagent disk.

A reaction device 3, driven by driving device (not shown in the figure), and periodically rotated around an axis; the reaction device 3 includes: a reaction disk 3-1, and multiple fixtures for cuvettes 3-2 are uniformly distributed along the circumference direction of the reaction disk, the fixtures for cuvettes 3-2 can be a circle structure component with several holes arranged around the circumference direction of the reagent disk, and are placed in the reagent disk for holding or placing cuvettes, for example, a tube holder, the number of cuvettes is 81, divided into 9 groups.

A sampling device 4, based on periodical rotation of the holding device 2 and the reaction device 3 and used for adding reagents and/or samples collected from the holding device 2 to the reaction device 3; the sampling device 4 can be selected as a sampling needle;

a driving system, connected with the holding device 2, the reaction device 3 and the sampling device 4 to control the holding device 2, the reaction device 3 and the sampling device 4 to perform corresponding operations.

a hydraulic device 5, including vacuum pump 5-1, vacuum tank 5-2 and flow paths 5-3, wherein the flow paths include pipelines and valves installed on the pipelines, and are also connected with the sampling device and an automated cleaning device;

the vacuum pump 5-1 is used to regulate pressure in the vacuum tank 5-2 so that the pressure in the vacuum tank 5-2 reaches the preset pressure;

the vacuum tank 5-2 is used to control the operation of the flow paths under the preset pressure to operate the sampling device ascending, descending and rotating, and to make the liquid enter into or discharge from the automated cleaning device.

An automated cleaning device 6, connected with the hydraulic device 5, is used to clean the cuvettes in the reaction device 3 based on the control of the hydraulic device 5; the automated cleaning device 6 can be a cleaning needle, which can suck liquid into the holding space of the cleaning needle, then release it to a cuvette to clean the cuvette, then after cleaning drain the waste liquid from the cuvette to a wastewater tank (not shown in the figure), and the wastewater tank can be set outside the detection apparatus.

A stirring device 7, connected with the driving system for stirring the mixtures of reagents and samples, and homogenously stirring after each adding step is completed. Preferably, the stirring device can be a stirring needle.

A temperature control device 8, located below multiple fixtures of cuvettes, and used to control temperature of the cuvettes in the fixtures of cuvettes to keep the temperature of the cuvettes at the set experimental temperature. Specifically, the temperature control device 8 is a temperature control groove, which is a groove structure, so that 81 cuvettes can be located inside the groove structure to maintain the reaction temperature and the incubation temperature in the reaction process.

A photoelectric device 9, which is arranged on the upper surface of the housing 1 for controlling the light path and wavelengths, providing light for the reaction between samples and reagents, and continuously measuring the absorbance value of the samples. The photoelectric device is a photoelectric box.

An injector 10, which is arranged on the upper surface of the housing 1.

Wherein, there is a cover on the sample reagent disk to close the sample reagent disk for providing an experimental environment for TR activity detection; there are sampling holes for samples, sampling holes for working liquid/inhibitor solution and sampling holes for mixed reagents in the cover of the sample reagent disk; the first and second liquid-filling holes are in the reaction disk; the sampling holes for samples, sampling holes for working liquid/inhibitor solution, sampling holes for mixed reagents, the first and second liquid-filling holes are all located on the same circle centered on the sampling device, and the sampling device moves periodically along the circumference direction of the circle among sampling holes for samples, sampling holes for working liquid/inhibitor solution, sampling holes for mixed reagents, the first and second liquid-filling holes.

Specifically, the interval between the first and second liquid-filling holes can be arranged to less than the distance of the number of cuvettes in each group, preferably the distance of 7-11 cuvettes, more preferably the distance of 8 cuvettes.

The example of the present invention also includes a host computer, which is connected with the hydraulic device and the driving system to send operation instructions to the hydraulic device and the driving system, so that the driving system controls the holding device 2 and the reaction device 3 to rotate periodically. When sampling is required, the driving system receives the instructions from the host computer, and controls the rotation of the holding device. When the holding device rotates every grid, the fixtures for sample tubes, the fixtures for working solution/inhibitor solution, and the fixtures for mixed reagents will rotate to the corresponding positions of sampling holes for samples, working solution/inhibitor solution and mixed reagent in the cover of the sample reagent disk. The sampling device will descend to the fixtures for sample tubes, working solution/inhibitor solution, and mixed reagents under the control of the hydraulic device, further to collect samples, working solution/inhibitor solution, or mixed reagents from the sample tubes, the working solution/inhibitor solution bottles, or the mixed reagent bottles, and then rise under the control of the hydraulic device, move to the position of the first or second liquid-filling hole, descend under the control of the hydraulic device, to add the collected samples, working liquid/inhibitor or mixed reagents to the cuvette at the positions of the first or second liquid-filling hole. Meanwhile, the reaction device rotates one grid so that the first and second liquid-filling holes correspond to the next cuvettes to be added with liquid, respectively, and then continues to complete collection and liquid addition of the next cycle.

The holding device, which is used to accommodate samples and reagents and to rotate periodically around an axis driven by the driving system; the reaction device, which is used to accommodate cuvettes and experimental cups that rotate periodically around an axis driven by the driving system; the sampling device, which is based on periodical rotation of the holding device and the reaction device, and used for adding reagents and/or samples collected from the holding device to the reaction device; the driving system, separately connected with the holding device, the reaction device and the sampling device to control the holding device, the reaction device and the sampling device to perform operations.

The following describes in detail the detection method of the present invention with the detection apparatus:

It should be noted that when the method of the present invention is used to detect thioredoxin reductase activity in human peripheral blood, sometimes two groups of control group and test group need to be detected in order to compare and detect the accuracy and rapidity of the present invention.

The following method can be applicable for the test group as well as the control group, wherein the detection method includes:

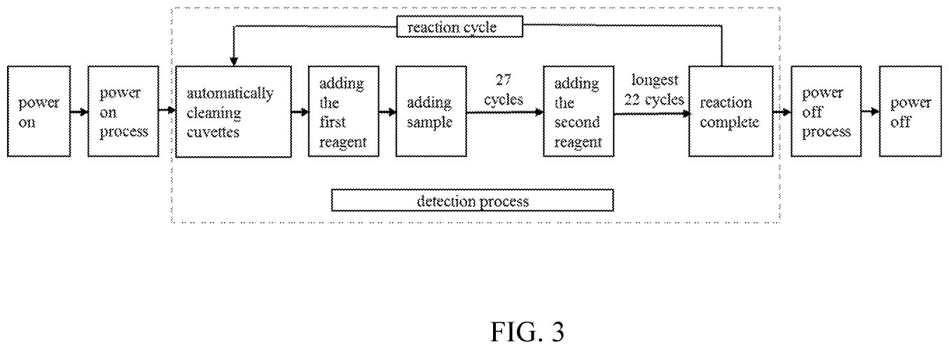

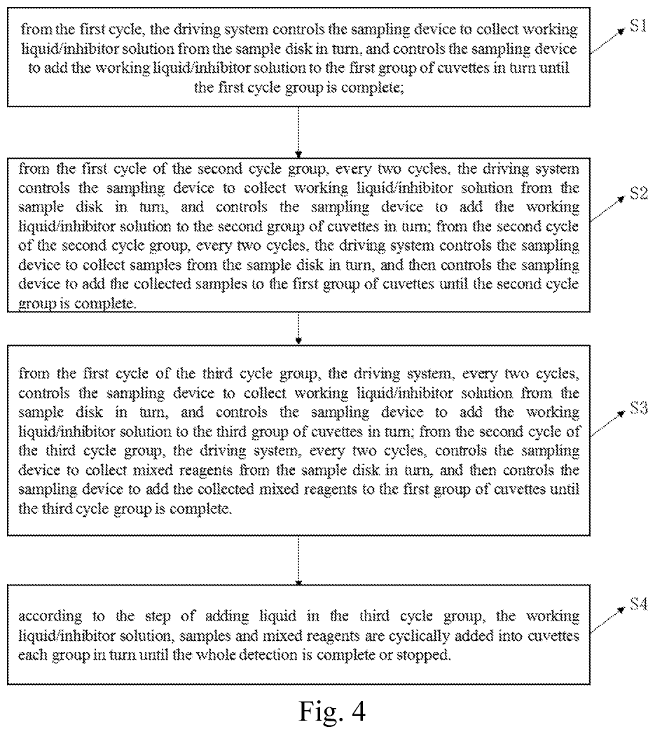

FIG. 3 and FIG. 4 show flow charts of the detection method for thioredoxin reductase activity in embodiments of the present invention.

As shown in FIG. 3 and FIG. 4, the detection method for thioredoxin reductase activity includes:

Step S1, from the first cycle, the driving system controls the sampling device to collect working liquid/inhibitor solution from the sample reagent disk in turn, and controls the sampling device to add the working liquid/inhibitor solution to the first group of cuvettes in turn until the first cycle group is completed;

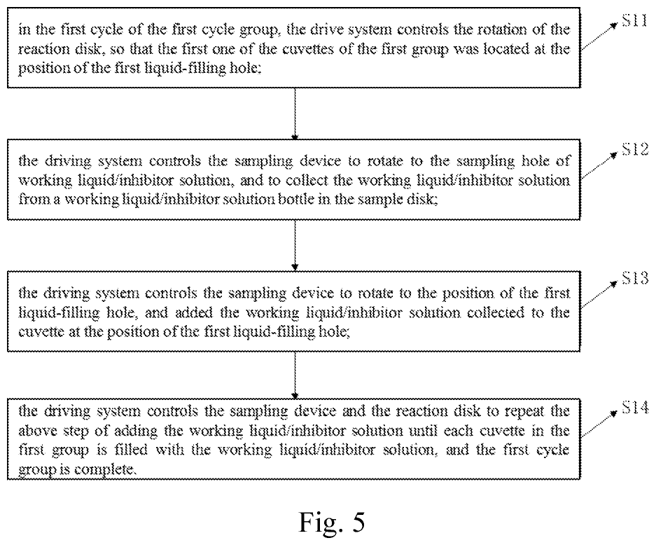

specifically, as shown in FIG. 5, step S1 included:

step S11, in the first cycle of the first cycle group, the drive system controls the rotation of the reaction disk, so that the first one of the cuvettes of the first group was located at the position of the first liquid-filling hole;

step S12, the driving system controls the sampling device to rotate to the sampling hole of working liquid/inhibitor solution, and to collect the working liquid/inhibitor solution from a working liquid/inhibitor solution bottle in the sample reagent disk;

step S13, the driving system controls the sampling device to rotate to the position of the first liquid-filling hole, and added the working liquid/inhibitor solution collected to the cuvette at the position of the first liquid-filling hole;

step S14, the driving system controls the sampling device and the reaction disk to repeat the above step of adding the working liquid/inhibitor solution until each cuvette in the first group is filled with the working liquid/inhibitor solution, and the first cycle group is completed.

Specifically, in the first cycle group, when the reaction disk rotates two preset angles, the sample reagent disk rotates one preset angle until the end of the first cycle group. Preferably, each preset angle can be set as the interval between fixtures of two cuvettes, so that each preset angle can be rotated and each cuvette in the first group of cuvettes is sequentially located at the position of the first liquid-filling hole;

step S2, from the first cycle of the second cycle group, every two cycles, the driving system controls the sampling device to collect working liquid/inhibitor solution from the sample reagent disk in turn, and controls the sampling device to add the working liquid/inhibitor solution to the second group of cuvettes in turn;

from the second cycle of the second cycle group, every two cycles, the driving system controls the sampling device to collect samples from the sample reagent disk in turn, and then controls the sampling device to add the collected samples to the first group of cuvettes until the second cycle group is completed.

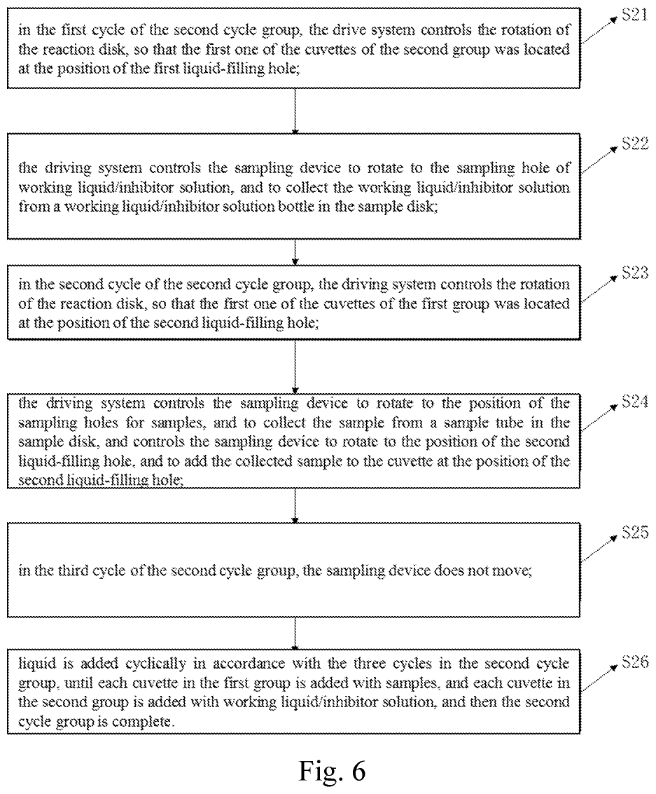

specifically, as shown in FIG. 6, step S2 includes:

step S21, in the first cycle of the second cycle group, the drive system controls the rotation of the reaction disk, so that the first one of the cuvettes of the second group was located at the position of the first liquid-filling hole;

step S22, the driving system controls the sampling device to rotate to the sampling hole of working liquid/inhibitor solution, and to collect the working liquid/inhibitor solution from a working liquid/inhibitor solution bottle in the sample reagent disk;

step S23, in the second cycle of the second cycle group, the driving system controls the rotation of the reaction disk, so that the first one of the cuvettes of the first group was located at the position of the second liquid-filling hole;

step S24, the driving system controls the sampling device to rotate to the position of the sampling holes for samples, and to collect the sample from a sample tube in the sample reagent disk, and controls the sampling device to rotate to the position of the second liquid-filling hole, and to add the collected sample to the cuvette at the position of the second liquid-filling hole;

step S25, in the third cycle of the second cycle group, the sampling device does not move; specifically, in the third cycle of the second cycle group, the reaction disk can rotate or not. If in the third cycle of the second cycle group it is chosen not to rotate, it can rotate in the next cycle.

However, considering the accuracy and convenience of the program control, the reaction disk is preferred not to rotate; and if the sample reagent disk needs to rotate in this cycle, it can be chosen to rotate in this cycle or in the next cycle.

Step S26, liquid is added cyclically in accordance with the three cycles in the second cycle group, until each cuvette in the first group is added with samples, and each cuvette in the second group is added with working liquid/inhibitor solution, and then the second cycle group is completed.

Step S3, from the first cycle of the third cycle group, the driving system controls the sampling device to successively collect working solution/inhibitor solution from the sample reagent disk every two cycles, and controls the sampling device to successively add the collected working solution/inhibitor solution to the cuvettes of the third group;

from the second cycle of the third cycle group, the driving system controls the sampling device to successively collect samples from the sample reagent disk every two cycles, and controls the sampling device to successively add the collected samples to the cuvettes of the second group; from the third cycle of the third cycle group, the driving system controls the sampling device to collect the mixed reagents from the sample reagent disk every two cycles, and controls the sampling device to add the collected mixed reagents to the cuvettes of the first group in turn until the third cycle group is completed.

When the third cycle group is completed, adding liquid to the cuvettes of the first group is completed and it starts incubation.

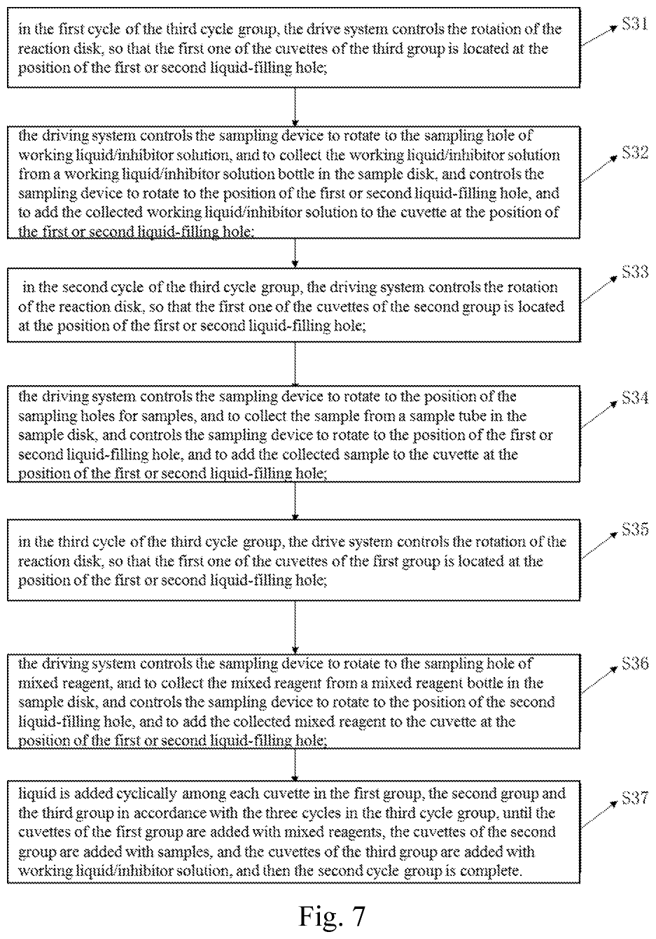

Specifically, as shown in FIG. 7, step S3 includes:

step S31, in the first cycle of the third cycle group, the drive system controls the rotation of the reaction disk, so that the first one of the cuvettes of the third group is located at the position of the first or second liquid-filling hole;

step S32, the driving system controls the sampling device to rotate to the sampling hole of working liquid/inhibitor solution, and to collect the working liquid/inhibitor solution from a working liquid/inhibitor solution bottle in the sample reagent disk, and controls the sampling device to rotate to the position of the first or second liquid-filling hole, and to add the collected working liquid/inhibitor solution to the cuvette at the position of the first or second liquid-filling hole;

step S33, in the second cycle of the third cycle group, the driving system controls the rotation of the reaction disk, so that the first one of the cuvettes of the second group is located at the position of the first or second liquid-filling hole;

step S34, the driving system controls the sampling device to rotate to the position of the sampling holes for samples, and to collect the sample from a sample tube in the sample reagent disk, and controls the sampling device to rotate to the position of the first or second liquid-filling hole, and to add the collected sample to the cuvette at the position of the first or second liquid-filling hole;

step S35, in the third cycle of the third cycle group, the drive system controls the rotation of the reaction disk, so that the first one of the cuvettes of the first group is located at the position of the first or second liquid-filling hole;

step S36, the driving system controls the sampling device to rotate to the sampling hole of mixed reagent, and to collect the mixed reagent from a mixed reagent bottle in the sample reagent disk, and controls the sampling device to rotate to the position of the second liquid-filling hole, and to add the collected mixed reagent to the cuvette at the position of the first or second liquid-filling hole;

step S37, liquid is added cyclically among each cuvette in the first group, the second group and the third group in accordance with the three cycles in the third cycle group, until the cuvettes of the first group are added with mixed reagents, the cuvettes of the second group are added with samples, and the cuvettes of the third group are added with working liquid/inhibitor solution, and then the second cycle group is completed.

Step S4, according to the step of adding liquid in the third cycle group, the working liquid/inhibitor solution, samples and mixed reagents are cyclically added into cuvettes each group in turn until the whole detection is completed or stopped;

specifically, liquid is added cyclically in accordance with the three cycles in the third cycle group, until all of cuvettes are added with working liquid/inhibitor solution, samples and mixed reagents, or the detection is stopped.

In the implementation of step S4, specifically from the first cycle of the fourth cycle group, every two intervals, working liquid/inhibitor solution is collected in turn and then successively added to the cuvettes of the fourth group; from the second cycle of the fourth cycle group, every two intervals, samples are collected in turn and then successively added to the cuvettes of the third group; from the third cycle of the fourth cycle group, every two intervals, mixed reagents are collected in turn, and successively added to the cuvettes of the second group.

For the reaction device, as long as the reaction device is working, the above step S3 can be repeated. It should be noted that after each group of cuvettes are added with working solution/inhibitor solution, samples and mixed reagents, the reaction starts. The longest reaction time is 22 cycles (22.5 s each cycle), preferably 20 cycles. For the cuvettes of this group, during 22 cycles, if the cuvettes rotate to the liquid-filling position, the sampling device is still in a waiting state. After the reaction of the cuvettes of this group is completed, instructions sent from the host computer can control the hydraulic device, and further control the automated cleaning device to remove waste liquid from the cuvettes and clean the cuvettes. After cleaning, the cuvettes of the group continue the operation of being added with working liquid/inhibitor solution, samples and mixed reagents.

For example, for the cuvettes of the first group, after the third cycle group is completed, a mixed solution of working liquid/inhibitor solution, sample and mixed reagent contained in the cuvettes of the first group starts to react. At this time, in the next cycle, if the cuvettes of the first group in the reaction disk rotate to the liquid-filling position, the sampling device is in a waiting state.

In the embodiment of the present invention, the cooperative detection device performs the corresponding test process, that 81 cuvettes are divided into nine groups, each reaction has 74 test cycles, including 27 cycles from adding the first reagent to adding a sample, 27 cycles from adding the sample to adding the second reagent, and 20 cycles of the reaction, and each cycle takes 22.5 seconds.

1) 1-9 cycles (22.5 s per cycle):

during each cycle of the 1-9 cycles, working liquid/inhibitor solution is collected in turn and successively added into the No. 1-9 cuvettes;

specifically, the sampling device collects the working liquid/inhibitor solution (working liquid or inhibitor solution) from the sampling hole position of working liquid/inhibitor solution, and adds it to the No. 1 cuvette in the reaction device. Then the reaction device rotates one grid and takes one cycle (22.5 s);

repeating the above movements 9 times continuously, and adding the working liquid/inhibitor solution into the No. 1-9 cuvettes (this is the first group), which totally takes 9 cycles, with the cuvette rotating 9 grids; wherein, when the reaction device rotates twice each time, the holding device rotates once. That is, the sampling device collects twice samples from each working liquid/inhibitor solution bottle in the sample reagent disk, and then the holding device rotates once.

2) 10-36 cycles (22.5 s per cycle):

During the 10th, 13th, 16th, 19th, 22nd, 25th, 28th, 31st and 34th cycles, samples are collected in turn and successively added to the 1-9 cuvettes;

during the 11st, 14th, 17th, 20th, 23rd, 26th, 29th, 32nd and 35th cycles, working liquid/inhibitor solution is collected in turn, and successively added to the 10-18 cuvettes;

during 12th, 15th, 18th, 21st, 24th, 27th, 30th, 33rd and 36th cycles, the sampling device does not move.

3) from 37 cycles:

From the 37th cycle, the working liquid/inhibitor solution is collected at three intervals, and successively added to the 19-27 cuvettes; from the 38th cycle, samples are collected at three intervals, and successively added to the 10-18 cuvettes; from the 39th cycle, mixed reagents are collected at three intervals, and successively added to the 1-9 cuvettes.

Specifically, in the 37th cycle, adding the working liquid/inhibitor solution to the 19th cuvette; in the 38th cycle, adding the sample to the 10th cuvette; in the 39th cycle, adding the mixed reagent to the 1st cuvette; in the 40th cycle, adding the working liquid/inhibitor solution to the 20th cuvette; in the 41st cycle, adding the sample to the 11th cuvette; in the 42nd cycle, adding the mixed reagent to the 2nd cuvette, and cycling accordingly until the whole detection is completed or stopped;

optionally, after the whole detection is completed, the cuvette group where the reaction has already finished can enter the next detection after cleaning.

During the whole detection process, the reaction disk can be set to rotate clockwise.

When the reaction disk rotates clockwise, the reaction disk rotates clockwise in each cycle of the first cycle group. For the first one in the cuvettes of first group, it can rotate clockwise or counterclockwise to the position of the first or second liquid-filling hole to be added with liquid. In the subsequent detection process, if the cuvettes to be added with liquid cannot rotate to the position of the first or second liquid-filling hole in accordance with a preset angle each cycle, it can rotate clockwise to the position of the two liquid-filling holes. Alternatively, it can rotate counterclockwise.

Through the above liquid-filling steps and the setting of cycle time, as well as the configuration of rotation between the two disks, the two disks work cooperatively. For the 1st cuvette, from adding samples to adding mixing reagents it waits for 27 cycles (about 10 minutes), which meets the medical requirements of TR activity detection; according to the above method, it realizes the detection of human blood samples, and the background deduction of the human blood samples with TR specific inhibitors, thus ensuring the consistency between the detection results of TR activity and the relevant national standards (TR detection data of healthy people is less than 4 units, and TR detection data of people highly associated with tumor is more than 12 units).

The detection method of the present invention for thioredoxin reductase activity can meet the requirements of automated detection by improving the preliminary detection method, and further has the following advantages:

1) compared with the disclosed detection method for TR activity (PCT/CN2010/078369), it significantly reduces incubation time that the incubation time of a single sample decreases from 30 minutes to 10 minutes, which can effectively reduce the detection time that the detection of every 40-50 samples can be completed in 1.5 hours, thus realizing the continuous detection of the cooperative detection apparatus and meeting the requirements of the detection throughout and speed of the cooperative detection apparatus for thioredoxin reductase activity.

2) Compared with the disclosed detection method for TR activity (PCT/CN2010/078369), it significantly reduces the movement steps of detection from 7 movement steps to 3-4 movement steps, which optimizes detection steps, reduces operation time of the apparatus and facilitates the operation of the apparatus program, so that a single tester can operate the apparatus independently and complete the whole detection process.

3) The detection method of the present invention can meet the requirements of movement, configuration instruction of the cooperative detection apparatus for thioredoxin reductase activity in TR clinical automation detection.

4) By the method of the present invention, the clinical TR activity detection can be completed with the cooperative detection apparatus for thioredoxin reductase activity, and processed by specific software (see another patent application: An analysis method and system of thioredoxin reductase activity), which can become the results of TR activity recognized by clinical medicine, and meet the corresponding product “TR Activity Assay Kit” on the market and the relevant requirements of national medical devices registration standards: YZB/country (Q/CVH 001-2011);

5) by using the existing “Thioredoxin reductase (TR) Activity Assay Kit” (certificates: Hubei Food and Drug Administration (approval) [2013] No. 2401815 and China Food and Drug Administration (approval) [2014] No. 3400264), the results of TR activity meet the relevant requirements of national medical devices registration standards: YZB/country (Q/CVH 001-2011).

As mentioned above, in the detection method for thioredoxin reductase activity provided in the present invention, by automatically sampling and mixing reagent A and reagent B in the automated cooperative detection apparatus to be a mixed reagent, and then automatically carrying out operations of adding it into the samples, mixing, and stirring in the automated cooperative detection apparatus, replacing the work of manually and repeatedly mixing and stirring the reagent A and reagent B which are separately added, thus improving work efficiency. Another object of the present invention is to provide an apparatus for biochemically detecting thioredoxin reductase (TR) in human blood, which is used to realize fully automated biochemical detection apparatus, and improve detection efficiency and save cost.

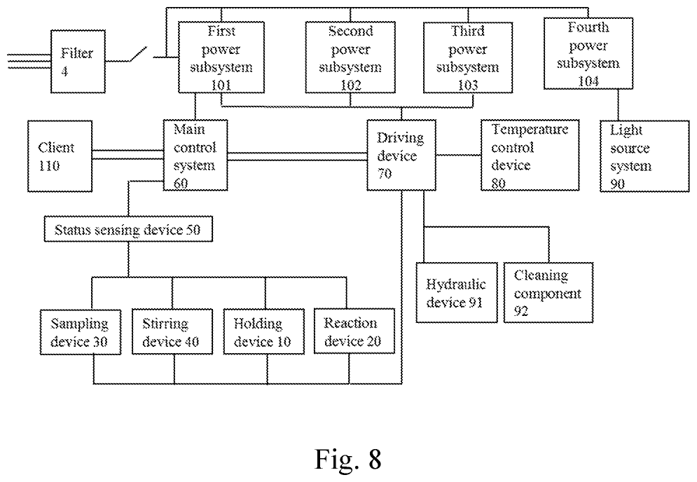

FIG. 8 is a schematic diagram of the system architecture of the biochemical detection apparatus for thioredoxin reductase (TR) of the present invention.

As shown in FIG. 8, the first embodiment of the present invention provides a biochemical detection apparatus, including: a holding device 10, a reaction device 20, a sampling device 30, a status sensing device 50, a main control system 60, a driving device 70.

The holding device 10, which is used to hold multiple reagents and/or samples, driven by a driving device 70, and periodically rotated around an axis, so that target reagents and/or samples are rotated to target liquid-filling holes.

The reaction device 20, which is used to hold multiple cuvettes, driven by the driving device 70, and periodically rotated around the axis, so that target cuvettes are rotated to target sampling holes.

The sampling device 30, which is driven by the driving device 70, periodically rotated around the axis and used for adding the target reagents and/or samples collected from the target sampling holes to the target cuvettes corresponding to the target liquid-filling holes.

The status sensing device 50, which is used for detecting the information of the reaction rotation status in the reaction device 20, the rotation status information of the holding in the holding device 10, and the reagent status information in the sampling device 30.

The main control system 60, which is connected with the status sensing device 50 and the driving device 70, respectively, and used for generating and sending corresponding control instructions to the driving device 70 based on the information of the reaction rotation status, the rotation status information of the holding, and the reagent status information.

The driving device 70, which is connected with the sampling device 30, the holding device 10 and the reaction device 20, respectively, and used for controlling the sampling device 30, the reaction device 20 and the holding device 10 to perform corresponding operations based on the received control instructions.

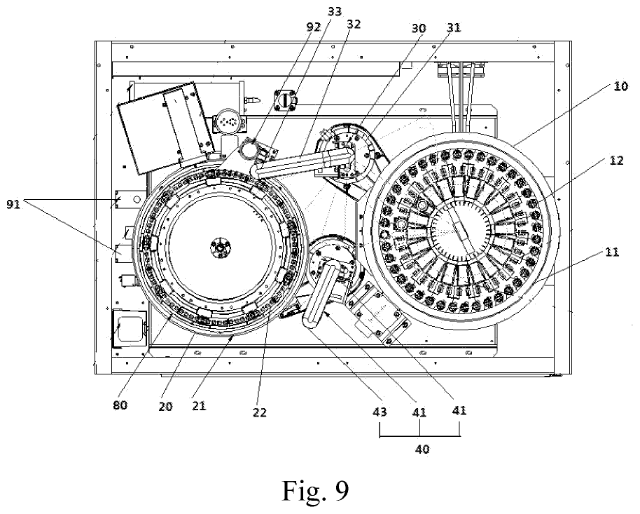

FIG. 9 is a schematic diagram of the mechanical structure of the biochemical detection apparatus for thioredoxin reductase (TR) of the present invention.

The holding device 10 includes: a holding disk 11, which is movably arranged and rotated periodically around the axis; there are multiple holding fixtures 12 in the holding disk 11, which are used for holding reagent bottles and/or sample bottles.

Optionally, the holding fixtures 12 are arranged on at least one circle of the holding disk 11, and each circle has a plurality of the holding fixtures 12. The number of the holding fixtures 12 can be chosen and arranged according to users' specific needs.

Preferably, a plurality of the holding fixtures 12 can be uniformly arranged along the edge of the holding disk 11, i.e., a predetermined distance between each two holding fixtures 12 can be arranged evenly to form at least one circle with the holding fixtures 12.

For each holding fixture 12, it can hold a reagent bottle seat or a sample bottle seat. When it is necessary to increase the number of sample positions and reduce the number of reagent positions, the reagent bottle seat can be removed and replaced with the sample bottle seat; when it is necessary to increase the number of reagent positions and reduce the number of sample positions, the sample bottle seat can be removed and replaced with the reagent bottle seat, realizing the flexible exchange between reagent positions and sample positions, which can meet customers' varying requirements for sample and reagent positions.

As shown in FIG. 9, the reaction device 20 includes: a reaction disk 21, which is movably arranged and rotated periodically around an axis and multiple fixtures for cuvettes 22, which are used for holding cuvettes.

Optionally, the holding fixtures 22 are arranged on at least one circle of the reaction disk 21, and each circle has a plurality of the reaction fixtures 22. The number of the reaction fixtures 22 can be chosen and arranged according to users' specific needs.

Preferably, a plurality of the reaction fixtures 22 can be uniformly arranged along the edge of the reaction disk 21, i.e., a predetermined distance between each two reaction fixtures 22 can be arranged evenly to form at least one circle with the reaction fixtures 22.

As shown in FIG. 9, the sample device 30 includes: a sampling rotating unit 31, which is movably arranged, and used to rotate periodically around an axis driven by the driving device 70; sampling fixtures 32, which are fixed on the sampling rotating unit 31 and driven to be rotated by the sampling rotating unit 31; a sampling needle 33, which has a fixed end on one end and a free end at the other end.

In combination with FIG. 8-9, the process of reagent addition is described:

the main control system 60, when receiving the information of the reaction rotation status that the target cuvettes are rotated to the target liquid-filling holes and the reagent status information that the collection of reagents is completed, generates an instruction of the addition of reagents; the driving device 70, after receiving the instruction of the addition of reagents, controls the sampling device 30 to rotate to the target liquid-filling holes of the reaction device 20 and to add the target reagents and/or the target samples to the target cuvettes corresponding to the target liquid-filling holes.

In combination with FIG. 8-9, the process of reagent sampling is described:

the main control system 60, when receiving the holding rotation status information that the target reagents and/or samples are rotated to the target sample holes and the reagent status information that the addition of the reagents is completed, generates the instruction for the collection of reagents;

the driving device 70, after receiving the instruction for the collection of reagents, controls the sampling device 30 to rotate to the target sample holes of the holding device 10 and to collect the target reagents and/or the target samples.

The third embodiment of the present invention provides a biochemical detection apparatus for thioredoxin reductase activity, also including: a stirring device 40, which is used to rotate periodically around an axis driven by the driving device 70 to stir mixed liquid formed in the target cuvettes.

As shown in FIG. 9, the stirring device 40 includes: a stirring rotating unit 41, which is movably arranged, and used to rotate periodically around an axis driven by the driving device 70; stirring fixtures 42, which are fixed on the stirring rotating unit 41 and driven to be rotated by the stirring rotating unit 41; a stirring needle 43, which has a fixed end on one end and a free end at the other end.

In combination with FIG. 8-9, the stirring process is described:

the main control system 60, when receiving the reagent status information that the addition of reagents is completed, generates a stirring control instruction;

the driving device 70, after receiving the stirring control instruction, controls the stirring device 40 to rotate to the target liquid-filling holes and to stir the mixtures in the target cuvettes.

In a biochemical detection apparatus for thioredoxin reductase activity provided in the fourth embodiment of the present invention, the status sensing device 50 is also used to detect the stirring status information of the stirring device 40; the main control system 60, after receiving the stirring status information that the stirring is completed, generates a stirring reset instruction; the driving device is connected with the stirring device 40 and controls the stirring device 40 to be reset.

In combination with FIG. 8-9, the process of reagent addition is described:

the main control system 60, when receiving the reaction rotation status information that the number of rotations by a predetermined angle is a predetermined number, generates a reagent rotation instruction; the holding device 10, after receiving the reagent rotation instruction, is controlled to rotate by the predetermined angle so that the next target cuvette is directed at the target liquid-filling hole.

The fifth embodiment of the present invention provides a biochemical detection apparatus for thioredoxin reductase activity, also including: a cleaning device 90, which is used to input liquid in cleaning liquid into the target cuvettes corresponding to the target liquid-filling holes for cleaning the target cuvettes.

As shown in FIG. 9, the cleaning device 90 includes a hydraulic device 91, which is used to control liquid in cleaning liquid entering target cuvettes or discharging waste liquid from the target cuvettes; a cleaning component 92, which is connected with the hydraulic device 91, under the action of the hydraulic device 91, to clean the target cuvettes.

The sixth embodiment of the present invention provides a biochemical detection apparatus for thioredoxin reductase activity, also including:

client 110, which is connected with the main control system 60, and used to provide an input interface for user operating instructions, and to collect the cleaning instruction input by the user on the input interface for user operation instructions, and send the cleaning instruction to the main control system 60; the driving device 70, after receiving the cleaning instruction, controls the cleaning device 90 to clean the target cuvettes corresponding to the target liquid-filling holes. The client 110 includes, but is not limited to, a host computer.

The sixth embodiment of the present invention provides a biochemical detection apparatus for thioredoxin reductase activity, also including:

a temperature control device 80, driven by the driving device 70, adjusts the experimental temperature of the reaction device 20 and keep the experimental temperature within the predetermined experimental temperature range.

In combination with FIG. 8-9, the process of temperature adjustment is described:

the status sensing device 50 is also used for collecting the experimental temperature data of the reaction device 20.

The main control system 60, after receiving the experimental temperature data, is used to analyze the experimental temperature data to obtain the current experimental temperature, and determine whether the current experimental temperature exceeds the predetermined experimental temperature range, if so, generate the control instructions of decreasing or increasing the temperature.

The driving system 70, after receiving the control instructions of decreasing or increasing the temperature, controls to reduce or raise the temperature, so as to keep the current experimental temperature in the predetermined experimental temperature range.

The seventh embodiment of the present invention provides a biochemical detection apparatus for thioredoxin reductase (TR) activity, also including: a light source system 90, which is used to provide predetermined experimental light conditions for cuvettes in the reaction device 20. The present invention can adopt the existing light adjustment technology, which will not be described here.

The eighth embodiment of the present invention provides a biochemical detection apparatus for thioredoxin reductase (TR) activity, also including: a power system 100, which is connected with the main control system 60, the driving device 70 and the light source system 90 to supply power for the main control system 60, the driving device 70 and the light source system 90.

The power system 100 includes: a first power subsystem 101, a second power subsystem 102, a third power subsystem 103, and a fourth power subsystem 104.

The first power subsystem 101 is connected in series with the main control system 60 and the drive device 70 to supply power to the main control system 60 and the drive device 70; the second power subsystem 102 is connected in series with the drive device 70 to supply power to the drive device 70; the third power subsystem 103 is connected in series with the drive device 70 and the temperature control device 80 to supply power to the drive device 70 and the temperature control device 80; the fourth power subsystem 104 is connected in series with the light source system 90 to supply power for the light source system 90. Wherein, the first power subsystem 101 is 5V DC, the second power subsystem 102 is 24V DC, the third power subsystem 103 and the fourth power subsystem 104 are 12V DC; the first power subsystem 101, the second power subsystem 102, the third power subsystem 103 and the fourth power subsystem 104 are in parallel.

The ninth embodiment of the present invention provides a biochemical detection apparatus for thioredoxin reductase (TR) activity, also including: a filter 4, which is electrically connected with the main control system 60, used for filtering the alternating current within the preset range input, and sending the filtered alternating current to the power system 100. Wherein, the alternating current within the preset range is 120 v-250 v.

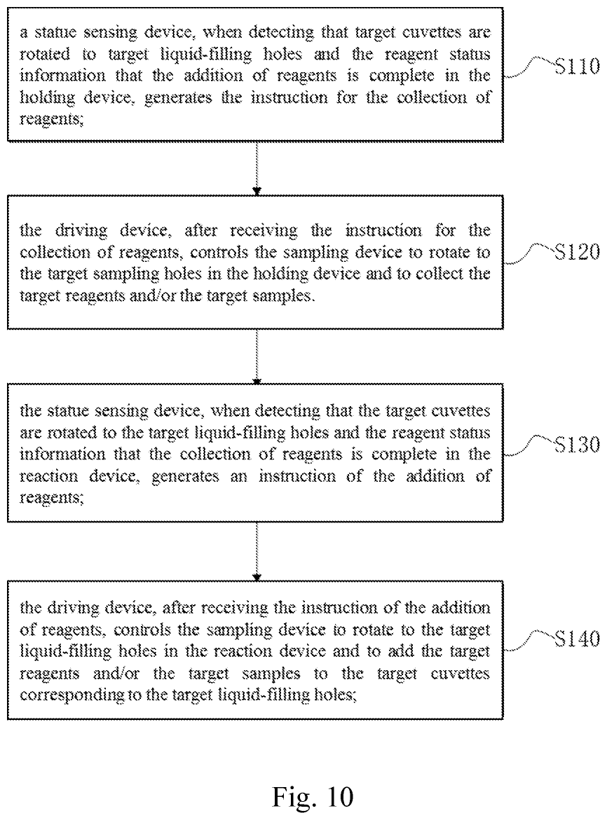

FIG. 10 is a flow chart of the operation method of the biochemical detection apparatus for thioredoxin reductase (TR) in the tenth embodiment in the present invention.

As shown in FIG. 10, an operation method of the biochemical detection apparatus for thioredoxin reductase activity includes:

step S110, a status sensing device, when detecting that target cuvettes are rotated to target liquid-filling holes and the reagent status information that the addition of reagents is completed in the holding device, generates the instruction for the collection of reagents;

step S120, the driving device, after receiving the instruction for the collection of reagents, controls the sampling device to rotate to the target sampling holes in the holding device and to collect the target reagents and/or the target samples;

step S130, the status sensing device, when detecting that the target cuvettes are rotated to the target liquid-filling holes and the reagent status information that the collection of reagents is completed in the reaction device, generates an instruction of the addition of reagents,

step S140, the driving device, after receiving the instruction of the addition of reagents, controls the sampling device to rotate to the target liquid-filling holes in the reaction device and to add the target reagents and/or the target samples to the target cuvettes corresponding to the target liquid-filling holes.

Here, steps S110 and S120 are the reagent addition processes. Steps S130 and S140 are the reagent collection processes. Generally, the reagents are collected first and then added, so step S120 is prior to step S140.

In the whole experimental process of the biochemical detection apparatus, reagents are collected first and added later. In the first collection, the signal that triggers the reagent collection is only that target reagents and/or target samples rotate to target sampling holes. After that, the signal that triggers the reagent collection is not only that the target reagents and/or the target samples rotate to the target sampling holes, but also that the reagent addition is completed.

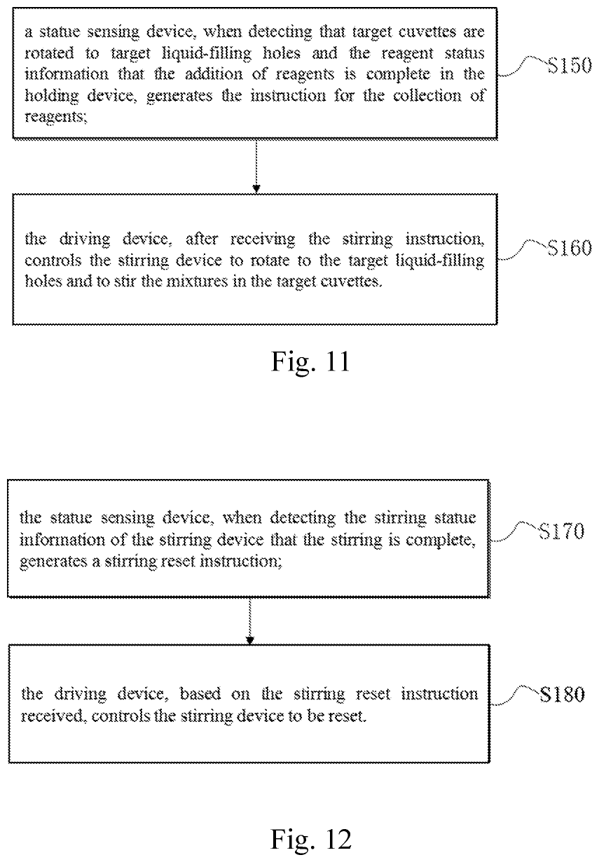

FIG. 11 is a flow chart of the operation method of the biochemical detection apparatus for thioredoxin reductase (TR) in the eleventh embodiment in the present invention.

As shown in FIG. 11, based on the tenth embodiment, the eleventh embodiment of the present invention provides an operation method of the biochemical detection apparatus for thioredoxin reductase (TR) activity, also includes:

step S150, a status sensing device, when detecting the reagent status information of the sampling device that the addition of reagents is completed, generates a stirring instruction;

step S160, the driving device, after receiving the stirring instruction, controls the stirring device to rotate to the target liquid-filling holes and to stir the mixtures in the target cuvettes.

FIG. 12 is a flow chart of the operation method of the biochemical detection apparatus for thioredoxin reductase (TR) in the twelfth embodiment in the present invention.

As shown in FIG. 12, based on the eleventh embodiment, the twelfth embodiment of the present invention provides an operation method of the biochemical detection apparatus for thioredoxin reductase (TR) activity, also includes:

step S170, the status sensing device, when detecting the stirring status information of the stirring device that the stirring is completed, generates a stirring reset instruction;

step S180, the driving device, based on the stirring reset instruction received, controls the stirring device to be reset.

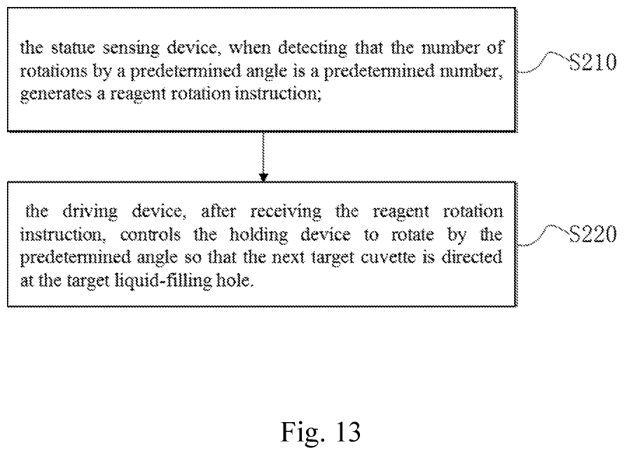

FIG. 13 is a flow chart of the operation method of the biochemical detection apparatus for thioredoxin reductase (TR) in the thirteenth embodiment of the present invention.

As shown in FIG. 13, the thirteenth embodiment of the present invention provides an operation method of the biochemical detection apparatus for thioredoxin reductase (TR) activity, also includes:

step S210, the status sensing device, when detecting that the number of rotations by a predetermined angle is a predetermined number, generates a reagent rotation instruction;

step S220, the driving device, after receiving the reagent rotation instruction, controls the holding device to rotate by the predetermined angle so that the next target cuvette is directed at the target liquid-filling hole.

the fourteenth embodiment of the present invention provides an operation method of the biochemical detection apparatus for thioredoxin reductase (TR) activity, also includes:

the driving device, when receiving the cleaning instruction output by the user on the input interface for user operation instructions, and based on said cleaning instruction received, controls said cleaning device to clean the target cuvettes corresponding to the target liquid-filling holes.

The working principle of the present invention is described in combination with the detection process of thioredoxin reductase activity in human blood:

In the present invention, there are 81 cuvettes in the reaction disk, which are divided into a control group and a test group. There are two divisions, in the first case, there are 40 cuvettes in the control group and 41 cuvettes in the test group; in the second case, there are 41 cuvettes in the control group and 40 cuvettes in the test group. 81 cuvettes are divided into 9 groups, each group including 9 cuvettes.

The holding disk has three circles of holding fixtures, and each circle holds 40 fixtures. Among them, the holding fixtures in the first circle hold the first reagent bottles, the holding fixtures in the second circle hold the sample bottles, and the holding fixtures in the third circle hold the second reagent bottles.

The final object of the present invention is to add the first reagents, samples and the second reagents to all 81 cuvettes. It should be noted that the first reagents, the second reagents and the samples in the embodiments of the present invention are all reagents related to the detection of thioredoxin reductase activity. Among them, the samples are blood, body fluids or tissue homogenates. The first reagents include working solutions (TrisHCl, morpholinopropanesulfonic acid, the mixed solution of disodium hydrogen phosphate/citric acid buffer and potassium dihydrogen phosphate/disodium hydrogen phosphate buffer) and thioredoxin reductase inhibitor compounds. The second reagents are mixed reagents, including a mixed solution of 5,5′-dithiobis(2-nitrobenzoic acid) or substituted 6,6′-dinitro-3,3′-dithiobenzoic acid and nicotinamide adenine dinucleotide phosphoric acid.

Wherein, the preparing process of the working solution: taking tri(hydroxymethyl)aminomethane hydrochloride (TrisHCl) (0.025-0.125 mol/L, pH 5.5.8-7.2), morpholinopropanesulfonic acid (0.25 mol/L), disodium hydrogen phosphate/citric acid buffer (0.2 mol/L) and potassium dihydrogen phosphate/disodium hydrogen phosphate buffer (1-15 mol/L) according to the ratio of 1:1:2:4; wherein, the pH of the disodium hydrogen phosphate/citric acid buffer is 2.2-8.0; the pH of the potassium dihydrogen phosphate/disodium hydrogen phosphate buffer is 4.9-8.2; wherein, mixing the working solution and the inhibitor in a ratio of 1:1-1:5 to form the inhibitor solution; mixing the inhibitor solution homogenously; wherein, the inhibitor is a thioredoxin reductase inhibitor compound, which could be a chemical monomer selenoline. Mixing the reagent A and the reagent B to form the mixed reagent in a ratio of 1:2-1:8; specifically, the range of the mixing ratios of reagent A and reagent B is 1:2-1:8, preferably 1:4-5, at which the mixed reagent formed by mixing for the detection provides higher accuracy, wherein, the reagent A is 5,5′-dithiobis(2-nitrobenzoic acid) or substituted 6,6′-dinitro-3,3′-dithiobenzoic acid; the reagent B is nicotinamide adenine dinucleotide phosphoric acid. For this part, please refer to another patent application “A detection method for thioredoxin reductase activity in human peripheral blood” by the same applicant.

As for the number of liquid-collecting holes, when only one reagent needs to be collected, it is only necessary to arrange one liquid-collecting hole in the cover of the holding disk; when a variety of reagents need to be collected, it is necessary to arrange multiple liquid-collecting holes in the cover of the holding disk, so that each reagent corresponds to a liquid-filling hole, the purpose of which is to speed up the liquid-collecting speed. In the detection experiment of thioredoxin reductase activity in human blood of the present invention, since three reagents need to be collected, there are three fluid-collecting holes arranged in the present invention, the first reagent corresponding to the first fluid-collecting hole, the sample corresponding to the second fluid-collecting hole, and the second reagent corresponding to the third fluid-collecting hole.

As for the number of liquid-filling holes, in order to improve the liquid-filling speed, there are two liquid-filling holes (the first liquid-filling hole and the second liquid-filling hole) arranged in the application. In the first cycle group, the first reagent is only added to the first group of cuvettes through the liquid-filling hole or the second liquid-filling hole. In the second cycle group, the sample is added to the first group of cuvettes, and the first reagent is added to the second group of cuvettes when the sample and the first reagent are added through the first and second liquid-filling holes. In the third cycle group, the second reagent is added to the first group of cuvettes, the sample is added to the second group of cuvettes, and the first reagent is added to the third group of cuvettes. After that, according to the liquid-filling sequence of the third group, the first reagent, the sample and the second reagent are further added to from the second group of cuvettes to the ninth group of cuvettes.

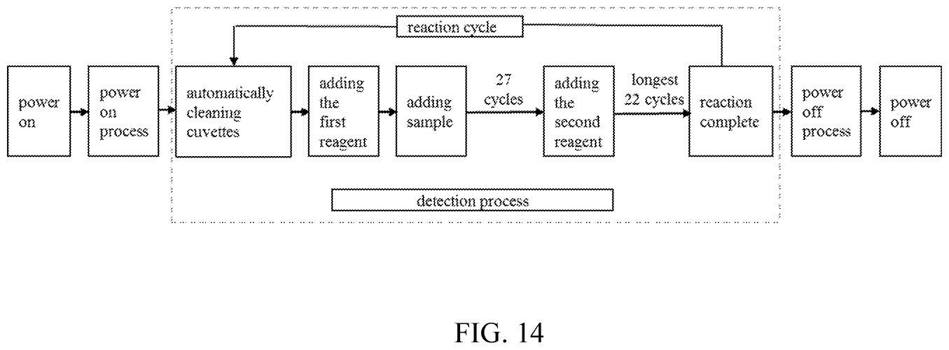

The experimental process for detecting thioredoxin reductase of the present invention is described in combination with FIG. 14:

Step S1, from the first cycle of the first cycle group, the driving device controls the rotation of the holding disk, when the status sensing device 50 detects that the first reagent in the holding device are rotated to the first or second liquid-filling hole and the reagent status information that the addition of the reagent is completed, and generates the instruction for the collection of the first reagent;

step S2, the driving device, which is based on the received instruction for the collection of the first reagent, controls the sampling device to rotate to the first sampling hole of the holding device to collect the first reagent;

step S3, when the status sensing device 50 detects that the first one in the first group of cuvettes is rotated to the first or second liquid-filling hole and the reagent status information of the sampling device 30 that the reagent collection is completed, the instruction for the addition of the first reagent is generated;

step S4, the driving device 70, after receiving the instruction for the addition of the first reagent, controls the sampling device 30 to rotate to the first or second liquid-filling hole of the reaction device 20 and to add the first reagent to the first cuvette corresponding to the first or second liquid-filling hole;

step S5, after the first cuvette in the first group of cuvettes is filled with the first reagent, it continues the above process of collecting and adding the first reagent, until all 9 cuvettes in the first group of cuvettes are all added with the first reagent, and the first cycle group is completed;

step S6, from the first cycle of the second cycle group, the driving device controls the rotation of the reaction device 20, when the status sensing device 50 detects that the first one in the second group of cuvettes in the reaction device is rotated to the first liquid-filling hole and the reagent status information of the sampling device that the reagent collection is completed, and generates the instruction for the collection of the first reagent;

S7, the driving device, after receiving the instruction of the addition of reagents, controls the sampling device to rotate to the first liquid-filling hole in the reaction device and to add the first reagent to the first cuvettes corresponding to the first liquid-filling hole;

S8, when the status sensing device detects the reagent status information of the reaction device that the reagent addition is completed, the instruction for the collection of the first reagent is generated;

S9, the driving device, after receiving the instruction for the collection of the first reagent, controls the sampling device to rotate to the first sampling hole of the holding device and to collect the first reagent.

S10, when the status sensing device detects that the first one in the second group of cuvettes is rotated to the first or second liquid-filling hole and the reagent status information of the status sensing device 50 that the reagent collection is completed, the instruction for the addition of the first reagent is generated;

S11, the driving device 70, after receiving the instruction for the addition of the first reagent, controls the sampling device 30 to rotate to the first liquid-filling hole of the reaction device 20 and to add the first reagent to the first cuvette corresponding to the first liquid-filling hole.

The sampling device collects the sample according to the above-mentioned process of collecting the first reagent, adds the first reagent in the first cycle, and adds the sample in the second cycle of the second cycle group in turn, until the first reagent is added to 9 cuvettes in the second cycle group, and the sample is added to 9 cuvettes in the first cycle group, and the second cycle group is completed.

In the first cycle of the third cycle group, the first reagent is added to the third group of cuvettes; in the second cycle, the sample is added to the second group of cuvettes; in the third cycle, the second reagent is added to the first group of cuvettes, until the first reagent is added to 9 cuvettes in the third group of cuvettes, the sample reagent is added to 9 cuvettes in the second group of cuvettes, and the second reagent is added to 9 cuvettes in the first group of cuvettes, and the third cycle group is completed.

According to the liquid-filling order of the third cycle group, the 4th-9th cuvettes are all added with the first reagent, the sample and the third reagent, until the detection of the whole disk is completed or stopped. For this part, please refer to another patent application “A cooperative detection apparatus and a detection method for thioredoxin reductase activity” by the same applicant.

It should be noted that after each group of cuvettes are added with the first reagent, the sample and the second reagent, the reaction starts. The longest reaction time is 22 cycles (22.5 s each cycle), preferably 20 cycles. For the cuvettes of this group, during 22 cycles, if the cuvettes rotate to the liquid-adding position, the sampling device is still in a waiting state. After the reaction of the cuvettes of this group is completed, instructions sent from the client can control the hydraulic device, and further control the automated cleaning device to remove waste liquid from the cuvettes and clean the cuvettes. After cleaning, the cuvettes of the group continue the operation of being added with the first reagent, the sample and the second reagent.

For example, for the cuvettes of the first group, after the third cycle group is completed, a mixed solution of the first reagent, the sample and the second reagent contained in the cuvettes of the first group starts to react. At this time, in the next cycle, if the cuvettes of the first group in the reaction disk rotate to the liquid-adding position, the sampling device is in a waiting state.

Because the biochemical detection apparatus of the present invention can detect not only biochemical reactions but also non-biochemical reactions, it only needs to input the parameters of biochemical reaction or non-biochemical reaction to be carried out on the user-input interface of the client, that is, it can automatically adjust according to the detection process of the specific biochemical reaction or non-biochemical reaction.

One object of the present invention is to provide a detection method for thioredoxin reductase activity, by setting the sample/reagent volume in the above automated cooperative detection apparatus which is applicable for thioredoxin reductase activity, the detection method can meet the requirements of the method for selecting the working liquid. When the method of the present invention is used in the above automated cooperative detection apparatus for thioredoxin reductase activity, it includes specified intelligent introduction methods for the driving system such as a method for operating sampling, and a method for the operational requirement of light shielding, a method for the operational requirement of mixing reagents, etc. The intelligent instructions such as the number of cycles in each cycle group and the time of each cycle specified in operation of the cooperative detection apparatus, and the requirements of the operation process are all related to the detection of TR function in human peripheral blood. The method of the present invention is a method applicable to the above cooperative detection apparatus for thioredoxin reductase activity for realizing the detection of the functional requirement of TR enzyme, and a detection method for thioredoxin reductase activity used in the above cooperative detection apparatus for thioredoxin reductase activity.

Another object of the present invention is also to protect a biochemical detection apparatus and its operation method for biochemically detecting thioredoxin reductase activity in human blood. For one thing the biochemical detection apparatus of the present invention realizes the automated detection of thioredoxin reductase activity in human blood for the first time, and solves the problem of the manual detection of thioredoxin reductase activity in the prior art; for another the biochemical detection apparatus of the application can not only realize the whole TR detection process of a single sample, but also realize the continuous TR detection process of multiple samples.

Specifically, 1) by arranging hardware settings, including the configuration of reaction disk, the configuration of accommodation disk, the linkage between the two disks, sample adding and sampling device, etc., the completion of the whole detection process of a single sample can be achieved. Due to the strict requirements on the continuous processing of samples, the continuous sample adding and the reaction time, as well as the steps that when sample disk rotates every two grids, the reaction disk rotates one grid, thus allowing two samples to be taken from each sample tube and added to two cuvettes respectively for the data detection of the test group and the control group, a synchronous detection of a single sample can be completed.

2) by arranging hardware settings and improving the method, wherein, the hardware settings include the configuration of the reaction disk, the configuration of the sample disk, the linkage between two disks, sample adding and sampling device, etc., the completion of multi sample continuous detection processes can be achieved. The improvement of the method includes the scheduling such as rotation time, rotation interval, rotation distance, sampling sequence and sample adding time, etc., specified for the sample disk and reaction disk. Since each group of cuvettes needs to be added with solutions three times, in the process of adding solution to each group of cuvettes, the next group of cuvettes can be added at the same time, therefore, multiple samples can be detected continuously and circularly, thus reducing detection time.

In addition, the present invention adopts specific driving hardware to make the software and hardware work cooperatively, which can improve the clinical detection efficiency of TR and save costs. And the experimental results detected by the biochemical detection apparatus of the present invention can meet the requirements of national testing standards.

It should be understood that the above-mentioned specific embodiments of the present invention are only used for illustrative description or explanation of the principles of the present invention, and do not constitute a limitation of the present invention. Therefore, any modification, equivalent alternative, improvement, etc. made without departing from the spirit and scope of the present invention are intended to be included within the protection scope of the present invention. In addition, the appended claims of the present invention are intended to cover all changes and modifications falling within the scope and boundaries of the appended claims, or the equivalents of such scope and boundaries.

1. A detection method for thioredoxin reductase activity, wherein, comprising:
preparing liquid,
preparing a working solution, inhibitor solution and mixed reagent;
adding samples,
adding 50 μL-70 μL of said working solution to a cuvette of the control group;
adding 50 μL-70 μL of said inhibitor solution to a cuvette of the test group;
adding 10 μL-30 μL of a sample to the cuvette of the control group and the cuvette of the test group, respectively;
incubating,
in the dark, incubating said cuvette of the control group and said cuvette of the test group at 30° C.-40° C. for the first predetermined time;
testing,
adding 100 μL-150 μL of the mixed reagent to said cuvette of the control group and said cuvette of the test group, respectively;
at a predetermined wavelength, determining the absorbance values in the second predetermined time period.

2. The detection method according to claim 1, wherein, said steps for preparing working solution comprise:
taking tri(hydroxymethyl)aminomethane hydrochloride, morpholinopropanesulfonic acid, disodium hydrogen phosphate/citric acid buffer and potassium dihydrogen phosphate/disodium hydrogen phosphate buffer according to the ratio of 1:1:2:4;
mixing said tri(hydroxymethyl)aminomethane hydrochloride, said morpholinopropanesulfonic acid, said disodium hydrogen phosphate/citric acid buffer and said potassium dihydrogen phosphate/disodium hydrogen phosphate buffer homogeneously.

3. The detection method according to claim 1, wherein,
the pH of said tri(hydroxymethyl)aminomethane hydrochloride is 5.5-7.2, and the concentration of it is 0.025-0.125 mol/L;
the concentration of said morpholinopropanesulfonic acid is 0.25 mol/L;
the pH of said disodium hydrogen phosphate/citric acid buffer is 2.2-8.0, and the concentration of it is 0.2 mol/L; the pH of said potassium dihydrogen phosphate/disodium hydrogen phosphate buffer is 4.9-8.2, and the concentration is 1-15 mol/L.

4. The detection method according to claim 1, wherein, said steps for preparing an inhibitor solution comprise:
mixing said working solution and said inhibitor in a ratio of 1:1-1:5 to form said inhibitor solution;
mixing said inhibitor solution homogenously;
wherein, said inhibitor is a thioredoxin reductase inhibitor compound.

5. The detection method according to claim 1, wherein, said steps for preparing a mixed reagent comprise:
mixing reagent A and reagent B to form said mixed reagent in a ratio of 1:4-1:8;
mixing said mixed reagent homogenously;
said reagent A is 5,5′-dithiobis (2-nitrobenzoic acid) or substituted 6,6′-dinitro-3,3′-dithiobenzoic acid;
and said reagent B is nicotinamide adenine dinucleotide phosphoric acid.

6. The detection method according to claim 1, wherein, said predetermined temperature is 30° C.-40° C.

7. The detection method according to claim 1, wherein, said first predetermined time is 8-20 minutes.

8. The detection method according to claim 7, wherein, said first predetermined time is 10 minutes.

9. The detection method according to claim 1, wherein, said first predetermined wavelength is 405 nm-450 nm.

10. The detection method according to claim 1, wherein, said second predetermined time is 20-30 cycles.

11. A detection apparatus for thioredoxin reductase activity, wherein, comprising:
a holding device (10), which is used to hold multiple reagents and/or samples, driven by a driving device (70), and periodically rotated around an axis, so that target reagents and/or samples are rotated to target liquid-filling holes;
a reaction device (20), which is used to hold multiple cuvettes, driven by the driving device (70), and periodically rotated around an axis, so that target cuvettes are rotated to the target sampling holes;
a sampling device (30), which is driven by the driving device (70), periodically rotated around an axis and used for adding said target reagents and/or samples collected from said target sampling holes to said target cuvettes corresponding to said target liquid-filling holes; a status sensing device (50), which is used for detecting the information of the reaction rotation status in said reaction device (20), the rotation status information of the holding in the holding device (10), the reagent status information in the sampling device (30);
a main control system (60), which is connected with said status sensing device (50) and said driving device (70), respectively, and used for generating and sending corresponding control instructions to the driving device (70) based on the information of the reaction rotation status, the rotation status information of the holding, and the reagent status information;
the driving device (70), which is connected with said sampling device (30), said holding device (10) and said reaction device (20), respectively, and used for controlling said sampling device (30), said reaction device (20) and said holding device (10) to perform corresponding operations based on the received control instructions.

12. The detection apparatus according to claim 11, wherein, said holding device (10) comprises:
a holding disk (11), which is movably arranged and rotated periodically around an axis;
multiple holding fixtures (12), which are used for holding reagent bottles and/or sample bottles.

13. The biochemical detection apparatus according to claim 1, wherein, said reaction device (20) comprises:
a reaction disk (21), which is movably arranged and rotated periodically around an axis;
multiple fixtures for cuvettes (22), which are used for holding cuvettes.

14. The detection apparatus according to claim 11, wherein, said sampling device (30) comprises:
a sampling rotating unit (31), which is movably arranged, driven by the driving device (70) and rotated periodically around an axis;
sampling fixtures (32), which are fixed on said sampling rotating unit (31) and driven to be rotated by said sampling rotating unit (31);
a sampling needle (33), which has a fixed end on one end and a free end at the other end.

15. The detection apparatus according to claim 11, wherein,
said main control system (60), when receiving the information of the reaction rotation status that the target cuvettes are rotated to the target liquid-filling holes and the reagent status information that the collection of reagents is completed, generates an instruction of the addition of reagents;
said driving device (70), after receiving the instruction of the addition of reagents, controls sampling device (30) to rotate to the target liquid-filling holes of the reaction device (20) and to add the target reagents and/or the target samples to the target cuvettes corresponding to the target liquid-filling holes.

16. The detection apparatus according to claim 11, wherein,
said main control system (60), when receiving the holding rotation status information that the target reagents and/or samples are rotated to the target sample holes and the reagent status information that the addition of the reagents is completed, generates the instruction for the collection of reagents;
said driving device (70), after receiving the instruction for the collection of reagents, controls the sampling device (30) to rotate to the target sample holes of the holding device (10) and to collect the target reagents and/or the target samples.

17. The detection apparatus according to claim 11, wherein, said biochemical detection apparatus also comprises:
a stirring device (40), which is driven by the driving device (70), periodically rotated around an axis, and used for stirring the mixtures formed in the target cuvettes.

18. The biochemical detection apparatus according to claim 17, wherein, said stirring device (40) comprises:
a stirring rotating unit (41), which is movably arranged, driven by the driving device (70) and rotated periodically around an axis;
stirring fixtures (42), which are fixed on a stirring rotating unit (41) and driven to be rotated by a stirring rotating unit (41);
a stirring needle (43), which has a fixed end on one end and a free end at the other end.

19. The detection apparatus according to claim 17, wherein,
said main control system (60), when receiving the reagent status information that the addition of reagents is completed, generates a stirring control instruction;
said driving device (70), after receiving the stirring control instruction, controls the stirring device (40) to rotate to the target liquid-filling holes and to stir the mixtures in the target cuvettes.

20. The detection apparatus according to claim 17, wherein,
said status sensing device (50) is also used to detect the stirring status information of the stirring device (40);
said main control system (60), after receiving the stirring status information that the stirring is completed, generates a stirring reset instruction.
the driving device is connected with the stirring device (40) and controls the stirring device (40) to be reset.

21. The detection apparatus according to claim 11, wherein,
said main control system (60), when receiving the reaction rotation status information that the number of rotations by a predetermined angle is a predetermined number, generates a reagent rotation instruction;
the holding device (10), after receiving the reagent rotation instruction, is controlled to rotate by the predetermined angle so that the next target cuvette is directed at the target liquid-filling hole.

22. The detection apparatus according to claim 11, wherein, further comprising:
a cleaning device (90), which is used to input the liquid in the cleaning liquid into said target cuvettes corresponding to said target liquid-filling holes for cleaning said target cuvettes.

23. The detection apparatus according to claim 22, wherein, said cleaning device (90) further comprises:
a hydraulic device (91), which is used to control the liquid in the cleaning liquid entering the target cuvettes or discharging the waste liquid from the target cuvettes.
a cleaning component (92), which is connected with said hydraulic device (91), under the action of said hydraulic device (91), to clean the target cuvettes.

24. The detection apparatus according to claim 22, wherein, further comprising:
client (110), which is connected with said main control system (60), and used to provide an input interface for user operating instructions, and to collect the cleaning instruction input by the user on said input interface for user operation instructions, and send the cleaning instruction to said main control system (60);
said driving device (70), after receiving the cleaning instruction, controls said cleaning device (90) to clean the target cuvettes corresponding to the target liquid-filling holes.

25. An operation method of the detection apparatus for thioredoxin reductase activity, wherein, comprising:
a status sensing device, when detecting that the target cuvettes are rotated to the target liquid-filling holes and the reagent status information that the collection of reagents is completed in the reaction device, generates an instruction of the addition of reagents;
the driving device, after receiving the instruction of the addition of reagents, controls the sampling device to rotate to the target liquid-filling holes in the reaction device and to add the target reagents and/or the target samples to the target cuvettes corresponding to the target liquid-filling holes;
the status sensing device, when detecting that the target cuvettes are rotated to the target liquid-filling holes and the reagent status information that the addition of reagents is completed in the holding device, generates the instruction for the collection of reagents;
the driving device, after receiving the instruction for the collection of reagents, controls the sampling device to rotate to the target sampling holes in the holding device and to collect the target reagents and/or the target samples.

26. The operation method according to claim 25, wherein, further comprising:
the status sensing device, when detecting the reagent status information of the sampling device that the addition of reagents is completed, generates a stirring instruction;
the driving device, after receiving the stirring instruction, controls the stirring device to rotate to the target liquid-filling holes and to stir the mixtures in the target cuvettes.

27. The operation method according to claim 26, wherein, further comprising:
the status sensing device, when detecting the stirring status information of the stirring device that the stirring is completed, generates a stirring reset instruction;
the driving device, based on the stirring reset instruction received, controls the stirring device to be reset.

28. The operation method according to claim 26, wherein, further comprising:
the status sensing device, when detecting that the number of rotations by a predetermined angle is a predetermined number, generates a reagent rotation instruction;
the driving device, after receiving the reagent rotation instruction, controls the holding device to rotate by the predetermined angle so that the next target cuvette is directed at the target liquid-filling hole.

29. The operation method according to claim 26, wherein, further comprising:
the driving device, when receiving the cleaning instruction output by the user on the input interface for user operation instructions, and based on said cleaning instruction received, controls said cleaning device to clean the target cuvettes corresponding to the target liquid-filling holes.
